(19) United States
(12) Patent Application Publication
(10) Pub. No.: US 2022/0370758 A1
(43) Pub. Date: Nov. 24, 2022

(54) METHOD AND SYSTEM FOR SYNTHESIZING BEAT SOUND AND MUSIC

(71) Applicant: Zhiwei WANG, Beijing (CN)

(72) Inventor: Zhiwei WANG, Beijing (CN)

(21) Appl. No.: 17/761,907

(22) PCT Filed: Oct. 28, 2019
(86) PCT No.: PCT/CN2019/113595

§ 371 Date: Mar. 18, 2022

(30) Foreign Application Priority Data
Sep. 18, 2019 (CN) 201910881204.3

(51) Int. Cl. A61M 21/02; G10H 1/00

(52) U.S. Cl. CPC A61M 21/02; G10H 1/0025; A61M 2021/0027

(57) ABSTRACT

A method and a system for synthesizing beat sound and music. The method comprises: selecting a sound material as a beat sound (S101); intelligently or manually regulating, according to a human body characteristic parameter, a beat sound frequency matched with the human body characteristic parameter (S102); selecting music (S103); and synthesizing the beat sound and the music in a volume ratio to obtain mixed music (S104). The method and system for synthesizing beat sound and music provided by the present disclosure can intelligently or manually regulate the beat sound frequency and the volume ratio of the beat sound and the music through human body characteristic parameter, and obtain the mixed music in various synthesis ways. The mixed music can be used in trainings for physical and mental relaxations and improving the concentration of the human body, and is suitable for practices of improving sleeping conditions of the human body, and improving practice effects of yoga, meditation, and zazen.

### TECHNICAL FIELD

The present disclosure relates to a technical field of music synthesis, and particularly to a method and a system for synthesizing beat sound and music.

### BACKGROUND

Generally, different sound elements have different influences on human emotions, some are exciting and some are sad. There are also some sound elements that have a calming effect, and if they are combined into beat sounds and repeated at beats suitable for human body rhythms, the effect of physical and mental relaxations will be more obvious.

However, if these beats are just used alone for emotion regulation, people will easily feel monotonous and boring and can hardly persevere, which makes it difficult to achieve an ideal effect. In this case, if soothing and graceful music is introduced as the background music and the beat sound is synthesized with the music in a specific way, the synthesized music will have good auxiliary effects on physical and mental relaxations and sleep improvement.

### SUMMARY

In order to achieve the above objective, the present disclosure provides a method and a system for synthesizing beat sound and music, which can intelligently or manually regulate a beat sound frequency and a volume ratio of the beat sound and the music according to human body characteristic parameters, and obtain mixed music by synthesizing in various synthesis ways. The sound effect and beat of the mixed music are more suitable for a current state of a human body, and suitable for practices of improving sleep conditions of the human body, and improving practice effects of yoga, meditation and zazen.

An embodiment of the present disclosure provides a method for synthesizing beat sound and music, comprising:

selecting a sound material as a beat sound;

intelligently or manually regulating, according to a human body characteristic parameter, a beat sound frequency matched with the human body characteristic parameter;

selecting music; and

synthesizing the beat sound and the music in a volume ratio to obtain mixed music.

Optionally, synthesizing the beat sound and the music in the volume ratio to obtain the mixed music comprises at least one of:

a system synthesis mode, which automatically synthesizes the beat sound and the music by a music synthesis module to obtain the mixed music;

a parallel synthesis mode, which plays the beat sound and the music respectively in a same physical space by a playing device, to obtain the mixed music by natural mixing; and

a creation synthesis mode, which performs and records the beat sound and the music directly through band performance and recording, or creation and recording by an electronic music system, to obtain the mixed music.

Optionally, the beat sound is a sound material played repeatedly or iteratively at a regular time interval; and the music is a music piece to be synthesized with the beat sound.

Optionally, the music is selected by:

receiving a manual selection instruction, and selecting music; or,

selecting music played recently or most frequently according to a user preference.

Optionally, the method further comprises:

collecting a human body characteristic parameter, wherein the human body characteristic parameter comprises at least one of a brainwave frequency, a heart rate and a respiratory frequency.

Optionally, intelligently or manually regulating, according to the human body characteristic parameter, the beat sound frequency matched with the human body characteristic parameter comprises:

determining, when the collected human body characteristic parameter comprises a brainwave frequency, whether the brainwave frequency is within a δ brainwave frequency range;

determining that a human body is in a sleep state and selecting a beat sound suitable for the sleep state or stop playing, if it is determined that the brainwave frequency is within the δ brainwave frequency range;

determining that the human body is in an awake state, if it is determined that the brainwave frequency is not within the δ brainwave frequency range;

further, selecting, if the human body characteristic parameter does not comprise a heart rate, a beat sound suitable for the awake state according to the brainwave frequency; and selecting, if the human body characteristic parameter further comprises a heart rate, a corresponding beat sound frequency according to the heart rate.

Optionally, intelligently or manually regulating, according to the human body characteristic parameter, the beat sound frequency matched with the human body characteristic parameter comprises:

selecting, when the collected human body characteristic parameter comprises the heart rate, a beat sound frequency proportional to the heart rate.

An embodiment of the present disclosure provides a system for synthesizing beat sound and music, comprising:

a beat sound selection module configured to select a beat sound from a sound material library, and intelligently or manually regulate, according to a human body characteristic parameter, a beat sound frequency matched with the human body characteristic parameter;

a music selection module configured to select music; and

a music synthesis module configured to synthesize the beat sound and the music in a volume ratio to obtain mixed music.

Optionally, synthesizing the beat sound and the music in the volume ratio to obtain the mixed music by the music synthesis module comprises at least one of:

a system synthesis mode, which automatically synthesizes the beat sound and the music by the music synthesis module to obtain the mixed music;

a parallel synthesis mode, which plays the beat sound and the music respectively in a same physical space by a playing device, to obtain the mixed music by natural mixing; and

a creation synthesis mode, which performs and records the beat sound and the music directly through band performance and recording, or creation and recording by an electronic music system, to obtain the mixed music.

Optionally, the system further comprises:

a human body characteristic parameter collection module configured to collect the human body characteristic parameter, which comprises at least one of a brainwave frequency, a heart rate and a respiratory frequency;

a light flashing module configured to emit flashing lights with a light flashing frequency the same as the beat sound frequency; and

a playing module configured to play the mixed music, wherein the playing module is configured to regulate the beat sound frequency, regulate a volume of sound, and turn off and wake up the system for synthesizing beat sound and music according to the human body state parameter.

The method and system for synthesizing beat sound and music provided by the present disclosure can intelligently or manually regulate the beat sound frequency and the volume ratio of the beat sound and the music through human body characteristic parameter, and obtain the mixed music in various synthesis ways. The mixed music can be used in trainings for physical and mental relaxations and improving the concentration of the human body, and is suitable for practices of improving sleeping conditions of the human body, and improving practice effects of yoga, meditation and zazen.

### BRIEF DESCRIPTION OF DRAWINGS

FIG. 1 is a flowchart of a method for synthesizing beat sound and music according to an embodiment of the present disclosure.

FIG. 2 is a schematic flowchart of selecting a beat sound according to an embodiment of the present disclosure.

FIG. 3 is a flowchart of a method for synthesizing beat sound and music according to another embodiment of the present disclosure.

FIG. 4 is a schematic diagram of an architecture of a system for synthesizing beat sound and music according to an embodiment of the present disclosure.

FIG. 5 is a schematic diagram of an architecture of a system for synthesizing beat sound and music according to another embodiment of the present disclosure.

FIG. 6 is a schematic diagram of a structure of a computer device according to an embodiment of the present disclosure.

### DESCRIPTION OF EMBODIMENTS

The principle and spirit of the present disclosure will be described below with reference to several exemplary embodiments. It should be understood that these embodiments are given only to enable those skilled in the art to better understand and realize the present disclosure, rather than limiting the scope of the present disclosure in any way. On the contrary, these embodiments are provided to make the present disclosure more thorough and complete, and to fully convey the scope of the present disclosure to those skilled in the art.

As known to those skilled in the art, an embodiment of the present disclosure can be implemented as a system, an apparatus, a device, a method or a computer program product. Therefore, the present disclosure can be embodied in the form of complete hardware, complete software (including firmware, resident software, microcode, etc.), or a combination of hardware and software.

According to an embodiment of the present disclosure, a method and a system for synthesizing beat sound and music are provided. The beat sound and the music may be selected based on human body characteristic parameters, and the beat sound and the music may be synthesized in various synthesis ways. The synthesized mixed music may be played to improve a concentration of a human body, which is suitable for practices of improving sleep conditions of the human body, and improving practice effects of yoga, meditation and zazen.

For different sound elements, some may have a calming effect on listeners, and for example may be sounds of ancient temple bells, ocean waves, musical instruments such as flute and clarinet, and chords synthesized with some notes. In the present disclosure, such sounds are combined into beat sounds, which are repeated at beats suitable for human body rhythms (human body characteristic parameter), and soothing and graceful music is introduced as the background music, so as to obtain mixed music by synthesis through a reasonable combination. Such mixed music can promote physical and mental relaxations and improve the concentration, and is suitable for practices of improving sleeping conditions of the human body, and improving practice effects of yoga, meditation and zazen. In addition, on the basis of the music synthesis, it is possible to enhance the effects with flashing lights.

Herein, the terms involved should be understood as follows.

Sound materials refer to those suitable for making beats and stored in a sound material library. These sound materials may be sounds of bells, pointer rotations, ocean waves, dripping water, musical instruments (such as flute and clarinet), and chords synthesized with notes. The sound material may be one syllable or a combination of a string of syllables composed of a plurality of syllables.

Beat sound refers to a sound material played repeatedly or iteratively at a regular time interval.

Source music refers to music to be synthesized with the beat sound. The music usually has the characteristics of soothing and calming, which can make listeners feel relaxed physically and mentally and improve sleep.

Beat sound frequency refers to the number of times that a beat sound appears per unit time.

Light flashing frequency refers to the number of times that a light flashes per unit time.

Volume ratio refers to a volume ratio between a beat sound and music.

Next, the principle and spirit of the present disclosure will be explained in detail with reference to several representative embodiments of the present disclosure.

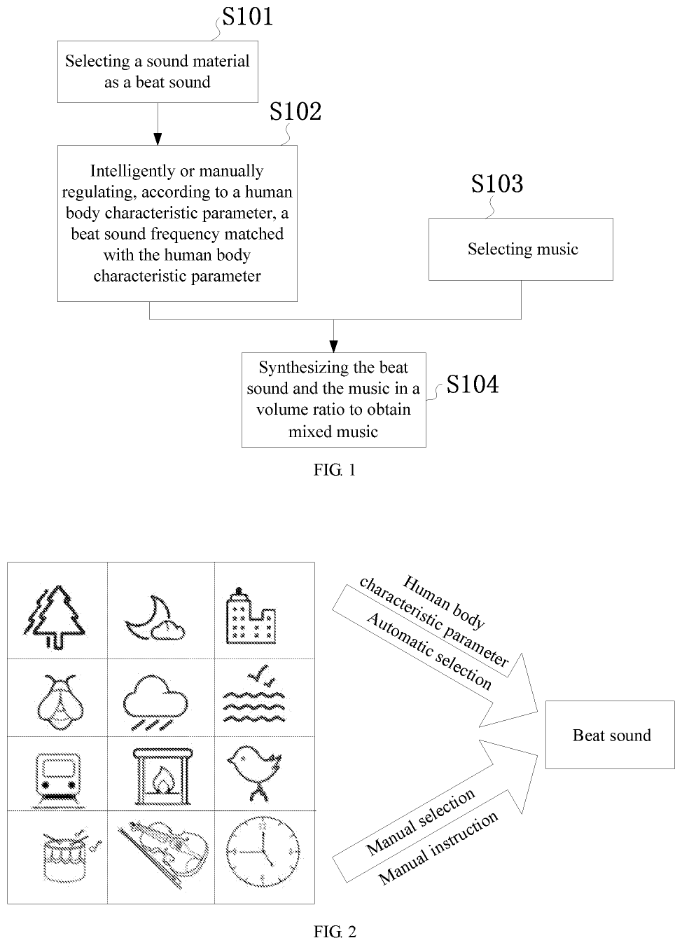

FIG. 1 is a flowchart of a method for synthesizing beat sound and music according to an embodiment of the present disclosure. As illustrated in FIG. 1, the method comprises:

S101: selecting a sound material as a beat sound. The method of selecting the beat sound may be an automatic selection or a manual selection. The manual selection may be subjectively selecting a sound material as a beat sound according to personal preferences, feelings, moods, or other factors. The automatic selection may be a random automatic selection, or may be made by analyzing playing history or user preference, etc.

S102: intelligently or manually regulating, according to a human body characteristic parameter, a beat sound frequency matched with the human body characteristic parameter.

Wherein, the sound materials may be sounds of bells, pointer rotations, ocean waves, dripping water, musical instruments (such as flute and clarinet), chords synthesized with notes, etc. According to step S101, a suitable one among the sound materials may be selected as the beat sound. Further, as illustrated in FIG. 2, referring to step S102, the beat sound may be selected by an automatic selection or a manual selection. The automatic selection is to select, according to a human body characteristic parameter, a beat sound matched with the human body characteristic parameter from a sound material library, and can intelligently regulate the beat sound frequency to match with the human body characteristic parameter. The manual selection is to select a beat sound from the sound material library based on a manually input selection instruction, and can regulate the beat sound frequency manually.

S103: selecting music.

In one embodiment, the music may be selected in a variety of ways, such as:

a first way, which selecting the music according to the manually input selection instruction; and

a second way, which selecting the music played recently or most frequently according to personal preferences; this selection process is an automatic selection, and mainly performed according to a user's historic record of playing music.

S104: synthesizing the beat sound and the music in a volume ratio to obtain mixed music, wherein the way of synthesizing in the volume ratio may be regulated intelligently according to the human body characteristic parameter or regulated according to a manual instruction.

In one embodiment, there are many synthesis ways, for example:

a system synthesis mode, which automatically synthesizes the beat sound and the music by a music synthesis module to obtain the mixed music. For example, the music synthesis module may be used to automatically synthesize the beat sound and the music to obtain the mixed music. In this mode, a music system may be used to select a specific beat sound from the sound material library, regulate a beat sound frequency according to the human body characteristic parameter, synthesize the beat sound with source music, and play the synthesized mixed music;

a parallel synthesis mode, which plays the beat sound and the music respectively in a same physical space by a playing device, to obtain the mixed music by natural mixing. In this mode, the source music and the beat sound are played independently from each other, and subjected to the natural mixing in the same physical space;

a creation synthesis mode, which performs and records the beat sound and the music directly through band performance and recording, or creation and recording by an electronic music system, to obtain the mixed music. In this mode, the mixed music, obtained directly through the band performance and recording, or the creation and recording by the electronic music system, has obvious beat sound elements and can be used for commercial promotions and applications, and also suitable for practices of improving sleep conditions of the human body and improving practice effects of yoga, meditation, and zazen.

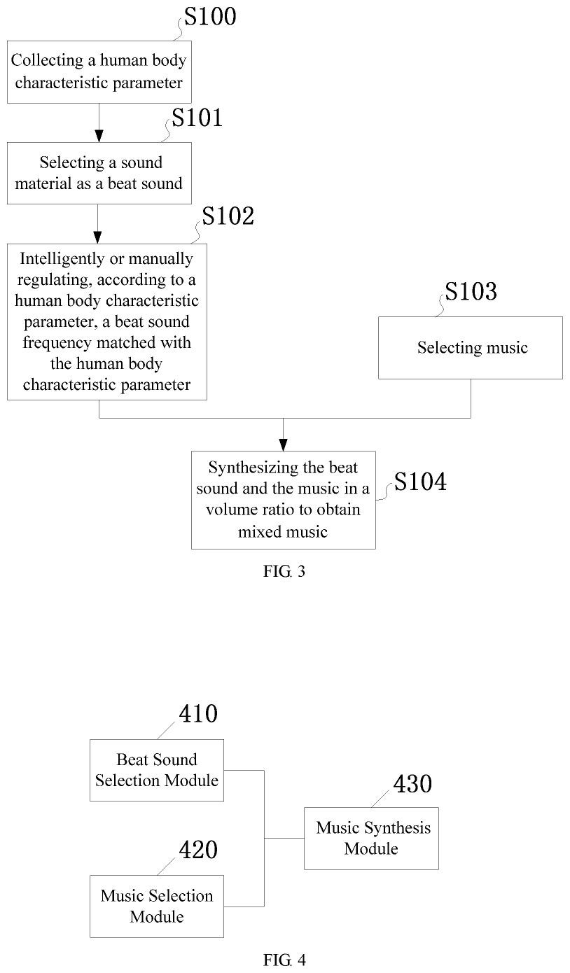

FIG. 3 is a flowchart of a method for synthesizing beat sound and music according to another embodiment of the present disclosure. As illustrated in FIG. 3, on the basis of the embodiment illustrated in FIG. 1, the method further comprises:

S100: collecting a human body characteristic parameter, wherein there may be one or more human body characteristic parameters, such as a brainwave frequency, a heart rate, and a respiratory frequency. It should be noted that the human body characteristic parameters may also comprise voice (e.g., speaking in a normal state, speaking in a dream, etc.), environmental noise, a motion frequency (e.g., a change of step number in Motion of WeChat), etc. These parameters may be collected by a human body wearable device such as a smartwatch, a smart bracelet, a mobile phone, etc. These indexes are quite different in different states of the human body.

Among these human body characteristic parameters, the heart rate and the brainwave frequency can best reflect the state of the human body. For example, the heart rate will become faster when people are moving; the heart rate will tend to be stable and slow when people are in a state of zazen and meditation; while the brainwave frequency will fall within a δ brainwave frequency range, i.e., 0.3 to 4 Hz, when people are in a sleep state.

However, the brainwave frequency will fall within a β brainwave frequency range (i.e., 12 to 25 Hz) or an α brainwave frequency range (i.e., 8 to 12 Hz), when people are in an awake state. The β brainwave is a conscious brainwave running at a frequency of 12 to 25 Hz. The β brainwave emits from the brain when people are in an awake state, attentive state, alert state, thinking state, analyzing, speaking or actively acting. The α brainwave runs at a frequency of 8 to 12 Hz, and emits when people are relaxed physically and mentally, in meditation, ‘daydreaming’ or in reverie. People having the a brainwave should be in a relaxed and awake state. As people fall asleep, the brainwave frequency will gradually decrease at a rate of 2 to 3 Hz per 5 to 10 minutes. During the decrease process, the brainwave frequency of 4 to 8 Hz falls within a θ brainwave frequency range. The θ brainwave runs at a frequency of 4 to 8 Hz and emits when people sink into fantasy or just go to sleep. At this time, people are in a ‘half-asleep’ hazy state in which the brain is processing information received during the day, and many inspirations may suddenly appear. Finally, the brainwave frequency will decrease to 0.3 to 4 Hz, i.e., within the δ brainwave frequency range. The δ brainwave runs at a frequency of 0.3 to 4 Hz and emits when people sleep deeply, while the frequency may fluctuate when people are dreaming.

Thus, in step S101, the specific process of selecting the beat sound matched with the human body characteristic parameter from a sound material library according to the human body characteristic parameter may be based on at least one of a brainwave frequency, a heart rate and a respiratory frequency.

The specific process may be as follows.

When the collected human body characteristic parameter comprises a brainwave frequency, it is determined whether the brainwave frequency is within a δ brainwave frequency range, and there may be three situations K1, K2, and K3 depending on the determination result.

K1: if it is determined that the brainwave frequency is within the δ brainwave frequency range, it is determined that the human body is in a sleep state, and a beat sound suitable for the sleep state is selected or the synthesis is stopped.

If it is determined that the brainwave frequency is not within the δ brainwave frequency range, it is determined that the human body is in an awake state, and there may be two further situations K2 and K3.

K2: if the collected human body characteristic parameter does not comprise the heart rate, i.e., only the brainwave frequency is collected, a beat sound suitable for the awake state may be selected according to the brainwave frequency; and if a person is in a going-to-sleep state, the beat sound will be regulated to be suitable for the sleep state along with the change of the brainwave frequency.

K3: if the collected human body characteristic parameter further comprises the heart rate, i.e., both the brainwave frequency and the heart rate are collected, a corresponding beat sound may be selected according to the heart rate. Specifically, a beat sound having a beat speed proportional to the heart rate can be selected according to the heart rate.

In addition, there may be another situation K4, i.e., the collected human body characteristic parameter comprises only the heart rate without any brainwave frequency. In this situation, a beat sound having a beat speed proportional to the heart rate is selected.

After the beat sound is selected, the source music to be synthesized with the beat sound may be further or simultaneously selected.

For any other human body characteristic parameters, such as the respiratory frequency, the beat sound and the music may also be selected in the above ways to meet the current state of the human body, which is not strictly limited herein. Any method that can implement the selection of the beat sound and the music based on the human body characteristic parameter should fall within the scope of the present disclosure.

To be noted, although the operations of the method of the present disclosure are described in a specific order in the above embodiments and drawings, it is not required or implied that these operations must be performed in the specific order, or that all of the operations illustrated must be performed to achieve the anticipated result. Additionally, or alternatively, some steps may be omitted, a plurality of steps may be combined into one step, and/or one step may be decomposed into a plurality of steps.

Based on the same inventive concept, the present disclosure further provides a system for synthesizing beat sound and music. As illustrated in FIG. 4, the system comprises:

a beat sound selection module 410 configured to select a beat sound from a sound material library, and intelligently or manually regulate, according to a human body characteristic parameter, a beat sound frequency matched with the human body characteristic parameter;

a music selection module 420 configured to select music; and

a music synthesis module 430 configured to synthesize the beat sound and the music in a volume ratio to obtain mixed music.

Optionally, synthesizing the beat sound and the music to obtain mixed music by the music synthesis module 430 may comprise at least one of the following modes, for example:

a system synthesis mode, which automatically synthesizes the beat sound and the music by the music synthesis module to obtain the mixed music;

a parallel synthesis mode, which plays the beat sound and the music respectively in a same physical space by a playing device, to obtain the mixed music by natural mixing; and

a creation synthesis mode, which performs and records the beat sound and the music directly through band performance and recording, or creation and recording by an electronic music system, to obtain the mixed music.

In another embodiment, as illustrated in FIG. 5, the system may further comprise:

a human body characteristic parameter collection module 440 configured to collect a human body characteristic parameter, which may comprise at least one of, for example, a brainwave frequency, a heart rate, and a respiratory frequency, as well as voice, environmental noise, or motion frequency, etc.;

a light flashing module 450 configured to emit flashing lights with a light flashing frequency the same as a beat sound frequency; the flashing lights at the same frequency as the beat sound can make people more engaged in yoga, meditation, zazen, and other practices, relax physically and mentally, and train and improve the concentration; and

a playing module 460 configured to play the mixed music, wherein the playing module is configured to regulate the beat sound frequency, regulate a volume of sound, and turn off and wake up the system for synthesizing beat sound and music according to the human body state parameter.

For example, during a process of a person going to sleep until entering deep sleep, the volume of sound may be regulated according to the parameters such as the brainwave frequency, the heart rate, the respiratory rate, the ambient sound, etc., so that the volume of sound gradually decreases and finally the music is turned off; and when the person wakes up, the mixed music may be played for awaking.

For another example, when a person is practicing yoga, if an action of the person is changed in amplitude and frequency, the human body characteristic parameters such as the heart rate and the respiratory rate will change. The mixed music may be regulated according to these changes, so that the mixed music can better meet the current state of the human body.

In one embodiment, the playing module 460 may be a home audio device, a headset, etc.

It should be noted that although several modules of the beat sound and music synthesis system are mentioned in the above detailed description, such division is only exemplary and not mandatory. Actually, according to the embodiments of the present disclosure, the features and functions of two or more modules described above may be embodied in one module. On the contrary, the features and functions of one module described above may be further divided to be embodied in a plurality of modules.

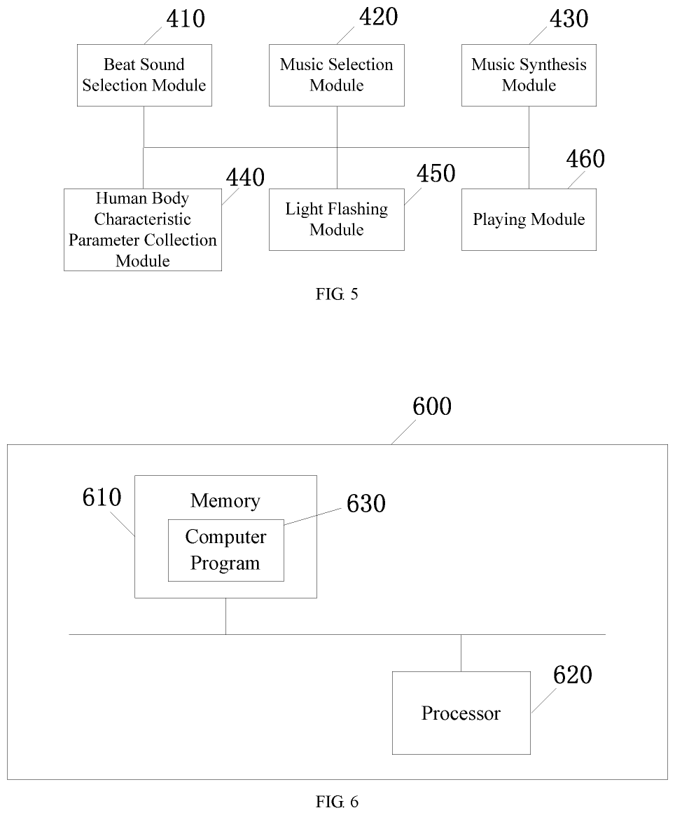

Based on the above inventive concept, as illustrated in FIG. 6, the present disclosure further provides a computer device 600, comprising a memory 610, a processor 620, and a computer program 630 stored in the memory 610 and executable on the processor 620, wherein the processor 620 is configured to execute the computer program 630 to implement the aforementioned method for synthesizing beat sound and music.

Based on the same inventive concept, the present disclosure further provides a computer-readable storage medium, which stores a computer program for implementing the method for synthesizing beat sound and music.

The method and system for synthesizing beat sound and music provided by the present disclosure can intelligently or manually regulate the beat sound frequency and the volume ratio of the beat sound and the music according to the human body characteristic parameters, and obtain the mixed music in various synthesis ways. The mixed music can be used in trainings for physical and mental relaxations and improving the concentration of the human body, and is suitable for practices of improving sleeping conditions of the human body, and improving practice effects of yoga, meditation, and zazen.

Although the spirit and principle of the present disclosure have been described with reference to several specific embodiments, it should be understood that the present disclosure is not limited to the disclosed specific embodiments. The division of various aspects does not mean that the features in those aspects cannot be combined for a benefit, but is only for the convenience of description. The present disclosure is intended to cover various modifications and equivalent arrangements included within the spirit and scope of the appended claims.

1. A method for synthesizing beat sound and music, comprising:
selecting a sound material as a beat sound, wherein the beat sound is a sound material played repeatedly or iteratively at a regular time interval, and the sound material is one syllable or a combination of a string of syllables composed of a plurality of syllables;
intelligently or manually regulating, according to a human body characteristic parameter, a beat sound frequency matched with the human body characteristic parameter, wherein the beat sound frequency is the number of times that the beat sound is played per unit time;
selecting music; and
synthesizing the beat sound and the music in a volume ratio to obtain mixed music.

2. The method for synthesizing beat sound and music according to claim 1, wherein synthesizing the beat sound and the music in the volume ratio to obtain the mixed music comprises at least one of:
a system synthesis mode, which automatically synthesizes the beat sound and the music by a music synthesis module to obtain the mixed music;
a parallel synthesis mode, which plays the beat sound and the music respectively in a same physical space by a playing device, to obtain the mixed music by natural mixing; and
a creation synthesis mode, which performs and records the beat sound and the music directly through band performance and recording, or creation and recording by an electronic music system, to obtain the mixed music.

3. The method for synthesizing beat sound and music according to claim 1, wherein the music is a music piece to be synthesized with the beat sound.

4. The method for synthesizing beat sound and music according to claim 1, wherein the music is selected by:
receiving a manual selection instruction, and selecting music; or,
selecting music played recently or most frequently according to a user preference.

5. The method for synthesizing beat sound and music according to claim 1, further comprising:
collecting a human body characteristic parameter, wherein the human body characteristic parameter comprises at least one of a brainwave frequency, a heart rate and a respiratory frequency.

6. The method for synthesizing beat sound and music according to claim 5, wherein intelligently or manually regulating, according to the human body characteristic parameter, the beat sound frequency matched with the human body characteristic parameter comprises:
determining, when the collected human body characteristic parameter comprises a brainwave frequency, whether the brainwave frequency is within a δ brainwave frequency range;
determining that a human body is in a sleep state and selecting a beat sound suitable for the sleep state or stop playing, if it is determined that the brainwave frequency is within the δ brainwave frequency range;
determining that the human body is in an awake state, if it is determined that the brainwave frequency is not within the δ brainwave frequency range;
further, selecting, if the human body characteristic parameter does not comprise a heart rate, a beat sound frequency suitable for the awake state according to the brainwave frequency; and selecting, if the human body characteristic parameter further comprises a heart rate, a corresponding beat sound frequency according to the heart rate.

7. The method for synthesizing beat sound and music according to claim 5, wherein intelligently or manually regulating, according to the human body characteristic parameter, the beat sound frequency matched with the human body characteristic parameter comprises:
selecting, when the collected human body characteristic parameter comprises the heart rate, a beat sound frequency proportional to the heart rate.

8. A system for synthesizing beat sound and music, comprising:
a beat sound selection module configured to select a beat sound from a sound material library, and intelligently or manually regulate, according to a human body characteristic parameter, a beat sound frequency matched with the human body characteristic parameter, wherein the beat sound is a sound material played repeatedly or iteratively at a regular time interval, and the sound material is one syllable or a combination of a string of syllables composed of a plurality of syllables, and wherein the beat sound frequency is the number of times that the beat sound is played per unit time;
a music selection module configured to select music; and
a music synthesis module configured to synthesize the beat sound and the music in a volume ratio to obtain mixed music.

9. The system for synthesizing beat sound and music according to claim 8, wherein synthesizing the beat sound and the music in the volume ratio to obtain the mixed music by the music synthesis module comprises at least one of:
a system synthesis mode, which automatically synthesizes the beat sound and the music by the music synthesis module to obtain the mixed music;
a parallel synthesis mode, which plays the beat sound and the music respectively in a same physical space by a playing device, to obtain the mixed music by natural mixing; and
a creation synthesis mode, which performs and records the beat sound and the music directly through band performance and recording, or creation and recording by an electronic music system, to obtain the mixed music.

10. The system for synthesizing beat sound and music according to claim 8, further comprising:
a human body characteristic parameter collection module configured to collect the human body characteristic parameter, which comprises at least one of a brainwave frequency, a heart rate and a respiratory frequency;
a light flashing module configured to emit flashing lights with a light flashing frequency the same as the beat sound frequency; and
a playing module configured to play the mixed music, wherein the playing module is configured to regulate the beat sound frequency, regulate a volume of sound, and turn off and wake up the system for synthesizing beat sound and music according to the human body state parameter.

11. The method for synthesizing beat sound and music according to claim 6, wherein intelligently or manually regulating, according to the human body characteristic parameter, the beat sound frequency matched with the human body characteristic parameter comprises:
selecting, when the collected human body characteristic parameter comprises the heart rate, a beat sound frequency proportional to the heart rate.
